(19) United States
(12) Patent Application Publication
(10) Pub. No.: US 2011/0250618 A1
(43) Pub. Date: Oct. 13, 2011

(54) BIOMARKERS AND ASSAYS FOR DIABETES

(75) Inventors: Randall W. Nelson, Phoenix, AZ (US); Chad R. Borges, Avondale, AZ (US); Paul Oran, Scottsdale, AZ (US)

(73) Assignee: Arizona Board of Regents, for and on behalf of Arizona State University, Scottsdale, AZ (US)

(21) Appl. No.: 12/922,403

(22) PCT Filed: Mar. 17, 2009
(86) PCT No.: PCT/US09/37369

§ 371 Date: Mar. 4, 2011

Related U.S. Application Data
(60) Provisional application No. 61/069,674, filed on Mar. 17, 2008.

(51) Int. Cl. G01N 33/566; G01N 33/53

(52) U.S. Cl. 435/7.21; 435/7.92

(57) ABSTRACT

The present invention is directed to novel biomarkers and combinations thereof. The present invention also provides assays and data evaluation methods related to the detection and monitoring of diseases, particularly, diabetes. In particular, the biomakers in accordance with the present invention include, but are not limited to, modified forms of nominally wild-type proteins, such as Gc-Globulin or GcG (also known as Vitamin D binding protein), beta-2-microglobulin (b2m), cystatin C (cysC), Albumin and Hem A&B. Particular forms of diabetes contemplated by the methods of the present invention include, but are not limited to, type 1 diabetes (T1D), type 2 diabetes (T2DM), pre-T1D and pre-T2DM. The present invention also provides methods of detecting multiple biomarkers in a single assay and to employ data evaluation methods that is able to accurately use these data in the determination and monitoring of diseases, such as diabetes.

### BACKGROUND OF THE INVENTION

It is estimated that diabetes—collective types 1 and 2 diabetes—afflicts nearly 24 million Americans, with nearly one third of these individuals unaware that they are affected by the disease. Diabetes is conservatively estimated to be the sixth leading cause of death in the U.S., and is found to occur disproportionately (in a greater percentage) in minority populations. The prevalence of diabetes, which has increased by ˜50% over the decade from 1990 to 2000, is estimated to double in the next forty years, and by many accounts is considered a pandemic threat within the nation with regards to increased mortality, decreased quality of life and escalating costs in healthcare. In 2007, it is estimated that the total cost of diabetes care was $174 billion, with a majority of that amount spent solely on medical expenditures. Diabetes is responsible for 12,000-24,000 new cases of blindness each year, and is the leading cause of kidney failure, responsible for ˜150,000 patients with end-stage kidney disease at a cost of >$ 7.5 billion/year for dialysis treatment alone. It is also responsible for 60% of non-traumatic lower limb amputations—82,000 in 2002 were due to diabetes—which, in a morbid view of cost accounting equates to the nation spending˜$8 billion each year to remove limbs. With regard to these major outcomes—death or disability—the effects of diabetes can be prevented (or at least delayed) through early detection and treatment. Non-drug treatment regimes focus on lifestyle intervention in the form of diet modification, weight loss and exercise regiments. Classical drug treatment of aggressive diabetes is through sulfonylureas or metformin, as well as formulations of short- and long-acting forms of insulin. More recently, new drugs, as typified by dipeptidyl peptidase IV inhibitors (e.g., Januvia and Galvus) have shown great promise in controlling blood glucose levels. Moreover, there are currently in excess of 350 drug candidates in development (e.g., GLP-1 analogs, DPP-IV inhibitors and SGLT2 inhibitors), making diabetes second only to cancer in health-related R&D focus. Important to the timely administration of all treatments is diagnosis at an early stage, preferably through the sensitive detection of biomarkers in easily accessible biofluids. Equally important—especially considering the many new drugs in development—is the use of markers to monitor the effectiveness of the treatments.

Currently, two biomarkers are commonly used in the detection of diabetes; blood glucose and glucose-modified hemoglobin (HbA1c). These two markers are essentially a direct (glucose) and indirect (HbA1c) monitor of elevated glucose in the blood stream. Each marker has its own usefulness in detecting and monitoring diabetes. Glucose is an immediate measurement of elevated blood glucose, and is used in both assisting diagnosis and monitoring of treatments for diabetes. HbA1c is a measurement of longer-term exposure to elevated blood glucose the time-scale is generally equated with the in vivo half-life of hemoglobin (60-90 days)—and is typically used in monitoring the ongoing management of diabetes. Both markers can be measured using a single clinical laboratory platform (e.g., Beckman Coulter SYNCHRON), although each requires a different assay scenario. Glucose is typically measured using enzyme assays (hexokinase) with spectrophotometric readout, whereas HbA1c is measured using a direct spectrophotometric measurement of total hemoglobin in combination with turbidimetric immunoinhibition for the measurement of the glycated hemoglobin. Additionally, a number of point-of-care devices have become available for both markers—e.g., Therasense Freestyle (glucose) and Bio-Rad in2it (HbA1c)—illustrating the importance of translating biomarkers and assays closer to the patient.

Both analyses rely on the accurate measurement of relatively small quantitative changes in the target biomarker. During fasting blood sugar tests, a blood glucose level of <100 mg/dL is considered normal, whereas levels greater than 126 mg/dL are consistent with diabetes; an approximately 25% change in concentration. Similar increases are associated with oral glucose tolerance tests (OGTT), where <140 mg/dL is considered normal and >200 mg/dL is indicative of diabetes (an ˜40% change). Instead of measuring an absolute concentration, glycated hemoglobin is measured relative to total hemoglobin. HbA1c values of <6% are the target values for normal individuals or diabetics undergoing treatment, whereas values greater than 7% are indicative of poor management and may warrant change in treatment (i.e., as little as a 16% change in relative abundance is considered significant). To compound matters, there are gray-areas in these values (i.e., fasting glucose of 100-125 mg/dL; OGTT=140-200 mg/dL; and HbA1c=6-7%), which are often attributed to a “pre-diabetic” state.

Accordingly, differentiating a healthy state from a pre-diabetic state or differentiating a pre-diabetic state from a diabetic state requires even more precise measurement than what the currently available single markers can provide.

As such, there is a need to develop multiple novel markers and assays, which when used with appropriate data evaluation methods are able to accurately detect diabetes as well as monitor the effects of treatment.

### SUMMARY OF THE INVENTION

The present invention identifies novel biomarkers and combinations thereof. The present invention also provides assays and data evaluation methods related to the detection and monitoring of diabetes. In particular, the biomarkers in accordance with the present invention include, but are not limited to, modified forms of nominally wild-type proteins. Modifications of proteins contemplated by the present invention can be conducted by methods well known in the art, including, but not limited to, genetic modifications (GM), posttranslational modifications (PTM) and/or metabolic alterations (MA). Particular forms of diabetes contemplated by the methods of the present invention include, but are not limited to, type 1 diabetes (T1D), type 2 diabetes (T2DM), pre-T1D and pre-T2DM. The biomarkers, assays and data evaluation methods also have implication in other disorders resulting in comparably modified forms of proteins. Of key importance is the ability of assays to unambiguously detect GM, PTM and MA forms of proteins while in the presence of the wild-type forms of the proteins. Additionally important is the ability to detect multiple biomarkers in a single assay and to employ data evaluation methods able to accurately use these data in the determination and monitoring of diabetes.

Accordingly, one aspect of the present invention is directed to novel biomarkers including, but not limited to, Gc-Globulin or GcG (also known as Vitamin D binding protein), beta-2-microglobulin (b2m), cystatin C (cysC), Albumin and Hem A&B.

Another aspect of the invention is directed to a method for the detection and monitoring of a disease or disorder, preferably, diabetes, by detecting and/assaying biomarkers including, but not limited to, GM, PTM and MA forms of human plasma and urinary proteins.

In still another aspect, the present invention is directed to a method for the detection and monitoring of a disease or disorder, preferably, diabetes, by using multiple assays to determine combinations of GM, PTM and/or MA related to diabetes.

In yet another aspect, the present invention is directed to a method for the detection and monitoring of a disease or disorder, preferably, diabetes, by using a single assay to simultaneously determine combinations of GM, PTM and/or MA related to diabetes.

In a particular aspect of the present invention, the GM, PTM and MA are all present on the same gene product and are all detected in a single protein-based analysis.

In still yet another aspect, multiple data obtained from the multiple markers in accordance with the methods of the present invention are further evaluated using classification algorithms to establish healthy and diabetic states.

In a further aspect, biomarkers in accordance with the methods of the present invention are correlated with in vivo lifetimes to establish a longitudinal record related to diabetic and pre-diabetic states.

In another particular aspect, biomarkers in accordance with the methods of the present invention are correlated with in vivo lifetimes to establish a longitudinal record related to the management and treatment of diabetes.

These and still further objects of the invention will become apparent upon reference to the following detailed description and attached drawings. To this end, various references are cited throughout the background section and detailed description, each of which is incorporated in its entirety herein by reference.

### BRIEF DESCRIPTION OF THE DRAWINGS

The file of this patent contains at least one drawing executed in color. Copies of this patent with color drawing(s) will be provided by the Patent and Trademark Office upon request and payment of the necessary fee.

FIG. 1. Overlays of deconvoluted ESI mass spectra resulting from the analysis of GcG from four individuals. The spectra are representative of data resulting from analysis of over 100 individuals investigated during the study. Indicated are signals from the three major allele products—Gc-1F, Gc-1S and Gc-2—as well as a low-frequency variant allele (‘variant’). Also observed is native glycosylation at Δm=+656 Da from their respective allele products (with the exception of Gc-2). The data are given to illustrate the extent of information resulting from the targeted “top-down” analysis of GeG when applied to populations.

FIG. 2. Allelic frequency of GcG in healthy (left columns, n=50 individuals) and T2DM (right columns; n=52 individuals). The Gc-1s allele is observed at approximately 5-fold greater incidence in the T2DM subjects.

FIG. 3. Mass spectral overlays of GcG from three individuals (all genotype Gc-1f/1f): healthy (in red), T2DM (green) and id-T2DM (blue), showing elevated glycated GcG related to T2DM. Inset: Box plot distribution of glycated GcG normalized to total GcG (integrals) for healthy (in red; n=50), T2DM (green; n=37) and id-T2DM (blue; n=15)—the dots represent the average values from the subjects. On average, diabetic individuals exhibited a 4-5 fold increase in relative glycation.

FIG. 4. Mass spectral overlays of b2m from three individuals: healthy (red), T2DM (green) and id-T2DM (blue), showing elevated levels of glycation related to T2DM. Inset: Box plot distribution of glycated b2m normalized to total b2m (integrated signals) for healthy (red; n=50), T2DM (green; n=37) and id-T2DM (blue; n=15). On average, diabetic individuals exhibited a 2-5-fold increase in relative signals due to glycation.

FIG. 5. Mass spectral overlays of cysC from the same samples used to generate FIGS. 3 & 4. Elevated glycation is indicated in T2DM (green) and id-T2DM (blue) relative to healthy (in red). Inset: Box plot distribution of glycated cysC normalized to total cysC (integrated signals) for healthy (in red; =50), T2DM (green; n=37) and id-T2DM (blue; n=15). On average, diabetic individuals exhibited a 3-4 fold increase in relative signals due to glycation.

FIG. 6. Mass spectral overlays of C-peptide from two individuals: healthy (in red) and T2DM (in green). A significantly higher relative presence of the des(GluAla) variant is observed in the T2DM individual (8.1%; MW=2819) compared to that of the healthy individual (1.7%; MW=2819). Inset: Box plots presenting the percent amount of des(GluAla) C-peptide respective to all isoforms present in the sample. The average for the healthy was 4.8% and for the T2DM it was 9.3%.

FIG. 7. Mass spectral overlays of TTR from the same samples used to generate FIG. 5. Elevated TTR sulfonation (relative to the native form of TTR) is indicated in T2DM (green) and id-T2DM (blue) relative to healthy (in red). Inset: Box plot distribution of the ratio of sulfonated to native TTR for healthy (in red; n=50), T2DM (green; n=37) and id-T2DM (blue; n=15). On average, diabetic individuals exhibited an approximate 10-fold increase in the sulfonated-to-native TTR ratio compared to healthy individuals.

FIG. 8. Relative glycation of b2m, cysC and GeG in 102 samples. The spatial separation of the 50 Healthy samples (red) from the 15 id-T2DM samples (blue) and 37 non-ID-T2DM samples (green) suggests that protein glycation biomarkers used in combination may serve to distinguish healthy patients from T2DM patients. Note that the GcG values are independent of genotype.

FIG. 9. Scores plot from principle components analysis of the 102 data points shown in FIG. 9—red points indicate healthy samples, blue points indicate ID-T2DM samples, and green points indicate non-ID-T2DM samples. Principle components 1 and 2 (plotted here) explain 94% of the variance observed in the raw data set and serve to generate a model for SIMCA-based classification.

FIG. 10. GcG genotype and GcG glycation summary with each data point resulting from a single analysis of GcG in healthy (red), T2DM (green) and id-T2DM (blue) individuals. Shaded dashed lines represent prophetic reference levels for genotype-dependent glycation as an indicator of T2DM—note, no healthy controls exhibited 1S/1S genotype so no value is given. Numbers (1-4) indicate values for individuals described in the text.

FIG. 11. Temporal monitoring using multiple MA. Values for relative glycation (Iglycation/Itotal×100) of each of the three markers are plotted versus in vivo half-life (into the past). Values connected by dashed lines are the average values obtained for healthy (Squares) T2DM (Inverted triangles) and id-T2DM (Circles) subjects. Individuals 1 (X's) and 2 (Forward Slash) demonstrate relatively good maintenance, especially several days before blood draw. Individuals 3 (Backward Slash) and 4 (Circle are observed to drift in and out of his/her respective categories, suggesting the need for more aggressive, or disciplined, therapy. Individual 5 (Triangles) demonstrates relatively good maintenance over several months.

FIGS. 12A-12D. Mass spectral overlays of (as indicated) Alb, Apo A1, Apo C1 and TTR taken from healthy and T2DM patients (Forward Slash). Insets-Box plot distributions showing % oxidation (measured per protein as the integral of glycated ion signal normalized to the integral of all species) healthy (n=50) and T2DM (Forward Slash; n=52).

FIG. 13 MSIA spectra of C-peptide from healthy and T2DM (Forward Slash).

FIG. 14. Positive-ion MSIA spectra of insulin from healthy and T2DM (Forward Slash) individuals.

FIG. 15. Receiver Operating Characteristic (ROC) curves for eight of the markers listed in Table 1, including S-Sulfonated TTR (S-Sulfonated TTR), APO C1 oxidized (Backward Slash), Glycated GcG (Triangle), Albumin oxidized (Inverted Triangle), Glycated Albumin (Square), Glycated CysC (X's), Glycated hemoglobin (Circles), Glycated B2m (Sinusoidal line) and Apo Ai oxidized (Sharp Wave Line).

FIG. 16. Plot of glycation versus oxidation generated from PC1 of glycation (from PCA using differential glycation values of four proteins) and PC1 of oxidation (generated from PCA of differential oxidation observed in three proteins). T2DM individuals are indicated with an X.

### DETAILED DESCRIPTION OF THE INVENTION

One embodiment of the present invention is directed to novel biomarkers including, but not limited to, Gc-Globulin or GcG (also known as Vitamin D binding protein), beta-2-microglobulin (b2m), cystatin C (cysC), Albumin and Hem A&B.

By “biomarker” is meant a substance used as an indicator of a biologic state. As used in the present application, a biomarker is a characteristic that can be objectively measured and evaluated as an indicator of normal biologic processes, pathogenic processes, or pharmacologic responses to a therapeutic intervention. A particularly preferred biomarker contemplated by the present invention is a substance whose detection indicates a particular disease or disorder state including, but not limited to, diabetes, cardiovascular disease, coronary and peripheral artery disease, chronic obstructive pulmonary disease, stroke, cancer, Alzheimer's disease, neuropathy, retinopathy and nutritional deficiencies; either alone or as comorbidities associated with diabetes. The present invention also contemplates a biomarker that indicates a change in expression or state of a protein that correlates with the risk or progression of a disease, or with the susceptibility of the disease to a given treatment.

According to the present invention, a biomarker can be genetically modified (GM), posttranslationally modified (PTM) or metabolically altered (MA). The contemplated biomarkers are found in gene products detected from common biological milieu (e.g., plasma, serum, urine, saliva, tears, sweat or tissue extracts). Genetic modifications can include, but are not limited to, nucleotide polypmorphisms, point mutations, haplotypes, allelic variants and splice variants. Posttranslational modifications include, but are not limited to, enzymatic and non-enzymatic modification of gene products related to general or specific physiologies. Metabolic alterations include, but are not limited to, enzymatic and non-enzymatic modification of gene products related to pathophysiologies of disease.

The present invention also contemplates assays and/or methods of data evaluation for use in the detection and monitoring of diseases or disorders including, but not limited to diabetes, cardiovascular disease, coronary and peripheral artery disease, chronic obstructive pulmonary disease, stroke, cancer, Alzheimer's disease, neuropathy, retinopathy and nutritional deficiencies; either alone or as comorbidities associated with diabetes. Preferably, the present invention is directed to assays and/or methods of data evaluation for use in the detection and monitoring of diabetes.

Accordingly, another embodiment of the invention is directed to a method for the detection and monitoring of a disease or disorder by detecting and/or assaying biomarkers including, but not limited to, GM, PTM and MA forms of human plasma and urinary proteins. The disease or disorder to be detected and/or monitored by the present invention include, but are not limited to, diabetes, cardiovascular disease, coronary and peripheral artery disease, chronic obstructive pulmonary disease, stroke, cancer, Alzheimer's disease, neuropathy, retinopathy and nutritional deficiencies; either alone or as comorbidities associated with diabetes. Preferably, the present invention is directed to method for the detection and monitoring of diabetes by detecting and/or assaying GM, PTM and MA forms of human plasma and urinary biomarker proteins.

Assays in accordance with the present invention can include both conventional or unconventional forms of gene product analysis, including but not limited to, immunometeric (e.g., enzyme-linked immunosorbent assay (ELISA), radioimmunoassay (RIA)), high performance liquid chromatography (HPLC), capillary electrophoresis (CE), 2-dimensional gel electrophoresis (2D-GE), surface plasmon resonance (SPR) and mass spectrometry (MS), or combinations thereof.

Methods of data evaluation in accordance with the present invention include, but are not limited to, linear regression, weighted and non-weighted evaluation of genotypic and phenotypic values, principal component analysis (PCA), soft independent modeling of class analogies (SIMCSA), and time-dependent evaluations, such as genotypic and phenotypic values versus disease state versus time (or protein half-life).

Detection and diagnosis of diabetes in accordance with the present invention include, but are not limited to, the determination risk factors and onset markers, and the combination thereof. Detection and diagnosis contemplated by the present invention also include the use of multiple markers in combination to accurately differentiate among a healthy, pre-diabetic and diabetic state, as well as differentiate a healthy, pre-diabetic or diabetic state from other diseases.

By “monitoring” in accordance with the present invention includes the use of one or more markers to ascertain the status or progression of diabetes, as well as response to treatment.

In still another embodiment, the present invention is directed to a method for the detection and monitoring of a disease or disorder, preferably, diabetes, by using multiple assays to determine combinations of GM, PTM and/or MA related to diabetes.

In yet another embodiment, the present invention is directed to a method for the detection and monitoring of a disease or disorder, preferably, diabetes, by using a single assay to simultaneously determine combinations of GM, PTM and/or MA related to diabetes.

In a particular embodiment of the present invention, the GM, PTM and MA are all present on the same gene product and are all detected in a single protein-based analysis.

In still yet another embodiment, multiple data obtained from the multiple markers in accordance with the methods of the present invention are further evaluated using classification algorithms to establish healthy and diabetic states.

In a further embodiment, biomarkers in accordance with the methods of the present invention are correlated with in vivo lifetimes to establish a longitudinal record related to diabetic and pre-diabetic states.

In accordance with the present invention, T2DM is detected and monitored by the following method. The method includes the following steps, resulting in the detection of specific proteins in a subject's body fluid. Plasma, serum, urine, saliva, tears, sweat or tissue extracts are all examples of suitable bodily fluids. Initially a fluid sample is collected from a subject. In one embodiment, the fluid sample collected is blood. After collection, the fluid is prepared to undergo Mass Spectrometric Immunoassay (MSIA) using electrospray ionization mass spectrometry (ESI-MS). The specific preparation and testing by MSIA utilizing ESI-MS is described more fully in Example 2 below.

In another embodiment, after collection the fluid is prepared to undergo MSIA using matrix-assisted laser desorption/ionization time-of-flight mass spectrometry (MALDI-TOFMS). The specific preparation and testing by MSIA utilizing MALDI-TOFMS is described more fully in Example 2 below.

Results provided by the specific mass spectrometer were collected for several glycation markers, including GcG, b2m, cysC, Alb, and Hem A&B. Further, results provided by the specific mass spectrometer were collected for several oxidative stress markers, including albumin (Alb), Apolipoprotein A1 (Apo A1), Apolipoprotein C1 (Apo C1), and transthyretin (TTR). Further, results provided by the specific mass spectrometer were collected for two Enzymatic signaling markers, including C-peptide (C-pep) and Insulin.

Glycation markers in T2DM subjects present themselves as positive mass shifts in MS results relative to target proteins of healthy subjects. This is further described in Example 4 below. Specifically, elevated proportions of glycation were observed in b2m, sysC, GcG, Alb and hemoglobin A&B chains ((Hem A&B)—a component of which is HbA1c).

Oxidative stress markers in T2DM subjects present themselves as positive mass shifts in MS results relative to target proteins of healthy subjects. This is further described in Example 8 below. Specifically, differential oxidation was observed in select high density lipoprotein components Apo A1, Apo C1 as well as TTR and Alb.

Enzyme markers in T2DM subjects, specifically C-peptide and Insulin, present themselves in a negative mass shift where certain proteins have been truncated. This is further described in Example 7 below. Specifically, truncated variants of C-pep and insulin were observed in greater abundance at higher frequency in T2DM subjects.

Initial univariate using receiver operating characteristics (ROC) and multivariate evaluation of all data with principal component analysis (PCA_ and soft independent modeling of class analogies (SIMCA) resulted in good separation between healthy and subjects with T2DM. This data can be used to monitor the trajectory of health to T2DM in a specific subject. Further this data can be used to provide a retrospective analysis of glycation levels for a subject over the past several days.

The present invention is further illustrated by the following non-limiting examples.

### Example 1

### Disease Subjects

### Healthy, type 2 Diabetes (T2DM) and Insulin-Dependent Type 2 Diabetes (id T2DM)

Given below are examples of genetic, posttranslational and metabolic alterations obtained through population screening of subjects consisting of healthy individuals (not known to have ailments; n=50), T2DM individuals (diagnosed as T2DM and treated through diet, exercise and non-insulin drugs; n=37) and id-T2DM individuals (insulin-dependent, diagnosed as T2DM and treated through administration of insulin; n=15). EDTA-plasma samples were collected from these individuals (under informed consent and IRB approval) after 8-hours fasting, and stored at −70° C. until analyzed using the methods described below. Records of gender, race, BMI, medical history and current treatment were also obtained for each diabetic individual.

### Example 2

### Population Proteomics and T2DM

Table 1 shows an exemplary list of 15 blood-borne markers (proteins & protein variants), each able to differentiate subjects between healthy and T2DM. It is important to note that all of the markers are due to the relative modulation of PTM's associated with physiological pathways known to be influential in the diagnosis or treatment of T2DM.

TABLE 1
Summary of Protein (Variant) Markers found in T2DM Subjects

| | | | ROC |
|---|---|---|---|
| | | Observation | (Area under |
| Protein | Category | (Ave: healthy vs T2DM) | curve) |
| Beta-2- | Glycation | 0.7 vs. 2.5%. | 0.84 |
| microglobulin | | | |
| Cystatin C | Glycation | 1.0 vs. 3.8% | 0.93 |
| GcG | Glycation | 0.9 vs. 4.8% | 0.98 |
| Albumin | Glycation | 13 vs 27% | 0.93 |
| Hem A&B | Glycation | 3.1 vs. 6.3% (b-chain) | 0.88-value |
| | | 1.7 vs. 3.4% (a-chain) | using all |
| | | 8.2 vs. 13.6% (b-chain; | Hemoglobin |
| | | +120 Da) | variants |
| Albumin | Oxidation | 40 vs. 25% | 0.96 |
| TTR | Oxidation | 1.5 vs. 30% | 0.99 |
| Apo A1 | Oxidation | 30 vs. 55% | 0.80 |
| Apo C1 | Oxidation | 8.8 vs. 28.9% | 0.98 |
| C-peptide | Enzymatic | 4.8 vs. 9.0% | 0.85 |
| Insulin | Enzymatic | 4.1 vs. 10.8 | 0.81 |

The hemoglobin MSIA detects HbA1c, as well as a second PTM of hemoglobin B-chain (at +120 Da) and glycation of the A-chain (+162 Da). Differential oxidation is monitored as depletion of the native form relative to all modified forms (e.g., cysteinylation at +119 Da). Differential glycation is also monitored (simultaneously) using this assay. Differential oxidation is increased sulfonation (+80 Da) occurring at cys10. Oxidation occurs at methionines (+16—to—+48 Da). Percentages reflect total oxidation capacity. Apo C1 has two forms, intact and truncated at n-terminal ThrPro. C-pep is truncated at n-terminal GluAla—termed C-peptide(3-31). Insulin is truncated at c-terminal Thr (b-chain). This assay also readily detects mass-shifted insulin formulations, e.g., Lantus and Novolog.

In the Observation Column of Table 1, the noted percentages are measures of specific species for each protein. Beta-2 microglobulin measures one form of relative glycation. Cystatin C measures one form of relative glycation. GcG measures one form of relative glycation and three haplotypes of genotype data which were correlated with T2DM. Albumin measures two forms of relative glycation and one form, cysteinylation, of oxidation. Hemoglobin A&B measure one form of relative glycation of hemoglobin A and two forms of hemoglobin B chains. TTR measures two forms of relative oxidation, cysteinylation and sulfonation. Apo A1 measures three forms of relative oxidation. Apo C1 measures two forms of relative oxidation. C-peptide measures two forms of relative truncations, des(E) and des(EA). Insulin measures one form of relative truncations of endogenous insulin, b-chain des (30) and relative contribution of administered forms of Novolog and Lantus and their truncated forms.

These investigations were performed using subjects consisting of 50 healthy individuals (i.e. these not known to have ailments), and 52 T2DM patients (comprised of 37 individuals diagnosed as T2DM and treated through diet, exercise and non-insulin drugs, and 15 insulin-dependent individuals who were diagnosed as T2DM and treated through administration of insulin). EDTA-plasma samples were collected from these individuals after 8-hours fasting, and stored at −70° C. until analyzed using the methods described below. Records of gender, race, BMI, medical history and current treatment were also obtained for each diabetic individual.

MSIA was performed using electrospray ionization mass spectrometry (ESI-MS) as follows. Human plasma samples (125 μL) were diluted 2-fold in HEPES-buffered saline (HBS) and placed in a 96-well titer plate. Proteins (and variants) were extracted using a robotic system fitted with extraction pipette tips prepared with rabbit anti-human polyclonal IgG toward the protein of choice. After extraction, non-specifically bound protein was removed through rinsing with HBS, water, 2M ammonium acetate/acetonitrile (3:1 v/v), then water again. Retained protein was next eluted by aspirating 5 μL of formic acid/acetonitrile/water (9/5/1 v/v/v) into the tips (covering the solid support) and after a short time (˜30 seconds) expelling the eluted protein into wells of a clean titer plate. Eluents were then diluted 2-fold with water in preparation for ESI-MS. Typically, 24 samples were processed in parallel (rather than the full 96) to match the daily throughput of the LC/ESI-MS. Mass spectrometry was performed using a Bruker microTOFq operating in conjunction with an Eksigent nanoLC*1D low-flow HPLC. A trap-and-elute form of sample concentration/solvent exchange rather than traditional LC was used for these analyses. Five-microliter samples were injected by a Spark Holland Endurance autosampler in microliter pick-up mode and loaded by the Eksigent nanoLC*1D at 10 μL/min (90/10 water/acetonitrile containing 0.1% formic acid, Solvent A) onto a protein captrap (Michrom Bioresources, Auburn, Calif.) configured for unidirectional flow on a 6-port divert valve. After two minutes, the divert valve position was automatically toggled and flow over the captrap cartridge was changed to 1 μL/min Solvent A (running directly to the ESI inlet) which was immediately ramped over 8 minutes to 10/90 water/acetonitrile containing 0.1% formic acid. By 10.2 minutes the run was completed and the flow back to 100% solvent A. Data were acquired in TOF-only mode by allowing all ions through the quadruple stage of the mass spectrometer (no preselection) and monitoring time-of-flight ions in the m/z range of 500-3000 (sampling at 5 kHz). Approximately 1.5 minutes of recorded spectra were averaged across the chromatographic peak apex of protein elution. The ESI charge-state envelope was deconvoluted with Bruker Daltonics' DataAnalysis v3.4 software to a mass range of 1000 Da on either side of any deconvoluted peak. Deconvoluted spectra were baseline subtracted and all peaks were integrated.

MSIA was performed using matrix-assisted laser desorption/ionization time-of-flight mass spectrometry (MALDI-TOFMS). Briefly, proteins and variants were extracted from plasma using a robotic system fitted with extraction pipette tips derivatized with rabbit anti-human polyclonal IgG toward the protein of interest. After extraction, non-specifically bound protein was removed through rinsing with HBS, water, 2M ammonium acetate/acetonitrile (3:1 v/v), then water again. Retained protein was next eluted by aspirating 5 μL of matrix solution (2:1 v/v, H₂O:ACN saturated with sinapinic acid with 0.4% added TFA) into the tips (covering the solid support) and depositing the matrix/protein mixture onto the surface of a 96-well formatted MALDI-TOF-MS target. Mass spectrometry was performed using a Bruker Autoflex III operating in delayed-extraction linear mode and laser (Nd:YAG) repetition rate of 200 Hz. Spectra (2,500 laser shots) were acquired by summing 25×100 laser-shot spectra [each meeting the criteria of S/N>10 and resolution (FWHM)>1,000] taken from different sites within a sample preparation. Spectra were processed by baseline subtraction followed by signal integration (to baseline) of each signal of interest. For each individual, the relative value of the variant (ion signal) was determined by normalizing the integral of the variant form of the protein to the integral of all observed forms of the protein.

### Example 3

### Gc-Globulin (aka Vitamin D Binding Protein)

### Genetic and Postranslational Modifications

Go-Globulin or GcG (also known as Vitamin D binding protein) is a plasma protein with a nominal molecular weight of ˜51 kDa and an estimated concentration in plasma of 200-600 mg/L. It is known to be present in human populations as three high-frequency allelic variants, Gc-1F, Gc-1S and Gc-2, as well as other low-frequency variants. Major biological roles for GcG include vitamin D metabolite transport, fatty acid transport, actin sequestration, and macrophage activation. Modification of this protein can thus constitute a biological event of wide-sweeping consequence.

During the course of investigation, genotypic and phenotypic variants of GcG were analyzed from blood plasma using immunoaffinity extraction followed by electrospray ionization mass spectrometry (ESI-MS). Human plasma samples (125 μL) were diluted 2-fold in HEPES-buffered saline (HBS) and placed in a 96-well titer plate. GcG (and variants) was extracted using the robotic system fitted with extraction pipette tips prepared with rabbit anti-human GcG polyclonal IgG. After extraction, non-specifically bound protein was removed through rinsing with HBS, water, 2M ammonium acetate/acetonitrile (3:1 vN), then water again. Retained protein was next eluted by aspirating 5 μL of formic acid/acetonitrile/water (9/5/1 v/v/v) into the tips (covering the solid support) and after a short time (˜30 seconds) expelling the eluted protein into wells of a clean titer plate. Eluents were then diluted 2-fold with water in preparation for ESI-MS. Typically, 24 samples were processed in parallel (rather than the full 96) to match the daily throughput of the ESI-MS. Mass spectrometry was performed using a Bruker microTOFq operating in conjunction with an Eksigent nanoLC*1D low-flow HPLC. A trap-and-elute form of sample concentration/solvent exchange rather than traditional LC was used for these analyses. Five-microliter samples were injected by a Spark Holland Endurance autosampler in microliter pick-up mode and loaded by the Eksigent nanoLC*1D at 10 μL/min (90/10 water/acetonitrile containing 0.1% formic acid, Solvent A) onto a protein captrap (Microm Bioresources, Auburn, Calif.) configured for unidirectional flow on a 6-port divert valve. After two minutes, the divert valve position was automatically toggled and flow over the captrap cartridge was changed to 1 μL/min Solvent A (running directly to the ESI inlet) which was immediately ramped over 8 minutes to 10/90 water/acetonitrile containing 0.1% formic acid. By 10.2 minutes the run was completed and the flow back to 100% solvent A. Data were acquired in TOF-only mode by allowing all ions through the quadruple stage of the mass spectrometer (no preselection) and monitoring time-of-flight ions in the m/z range of 500-3000 (sampling at 5 kHz). Approximately 1.5 minutes of recorded spectra were averaged across the chromatographic peak apex of GcG elution. The ESI charge-state envelope was deconvoluted with Bruker Daltonics' DataAnalysis v3.4 software to a mass range of 1000 Da on either side of any deconvoluted peak. Deconvoluted spectra were baseline subtracted and all peaks were integrated. Tabulated mass spectral peak areas were exported to a spreadsheet for further calculation and determination of relative peak abundances.

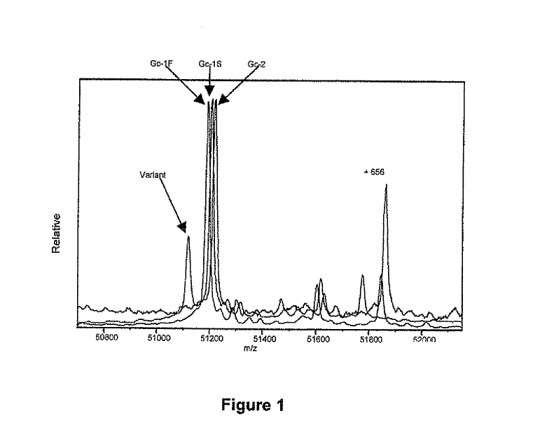

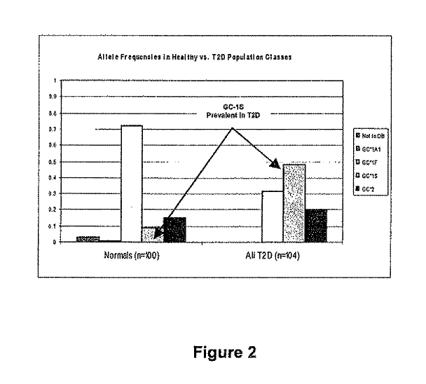

FIG. 1 shows overlays of deconvoluted ESI mass spectra resulting from the analysis of GcG from four individuals, which are given to illustrate the extent of information resulting from a single assay. Signals are observed for three homozygous genotypes that were commonly observed during the course of study. Indicated are Gc-1F (MW_{calc}=51188.2), Gc-1S (MW_{calc}=51202.2) and Gc-2 (MWcalc=51215.3 Da). The determined masses (for all samples analyzed in this manner) were within 2 Da of the calculated values. The three other genotypes that were observed at high frequency during study were heterozygous combinations of these three genotypes, i.e., Gc-1F/1S, Gc-1F/2 and Gc-1S/2. On occasion, other genotypic variants were observed throughout the study (indicated by variant), however, at low frequency within the populations under investigation. Also indicated are posttranslational modifications, namely O-linked glycosylation [(NeuAc)₁(Gal)1 (GalNAc)₁ trisaccharide]. Notably, the glycosylation signals were observed at consistent mass shifts relative (dm=+656 Da) to the Gc-1F and Gc-1S genotypes, but not the Gc-2 genotypes. This observation is consistent with the protein originating from the GcG-2 genotype lacking the preferred site of O-linked glycosylation (Thr⁴²⁰ changed to Lys⁴²⁰.) Upon evaluating only the genotyping data from all subjects (n=102 individuals), the Gc-1S allele (genotypes Gc-1S/1S, Gc-1F/1S and Gc-1S/2) was found predominantly in the T2DM subjects. As shown in FIG. 2, the allelic frequency increased by ˜500% in the T2DM subjects relative to the healthy subjects [Chi-squared test: (2 sample donor types×3 major GcG alleles; α=0.01; 2 degrees of freedom; χ2=49.6, p<0.0001; Cramer's V=0.474)].

This example demonstrates both genetic modifications (GM) and posttranslational modifications (PTM) present in products stemming from a single gene, and the ability to determine such modifications simultaneously using a single analysis (i.e., in a single analytical mode).

### Example 4

### Gc-Globulin (aka Vitamin D Binding Protein)

### Metabolic Alterations

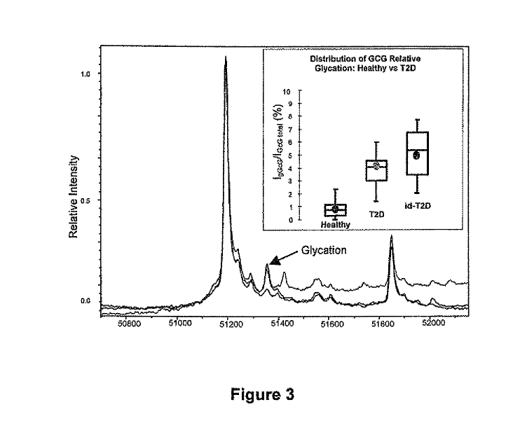

A particular advantage of protein-based analysis is the ability to map additional data not available through nucleic acid-based assays. As shown in FIG. 1, it is possible to further characterize GcG with regard to posttranslational modifications using the targeted ESI-MS assay. Notably, various protein phenotypes (posttranslational modifications), such as native glycosylation, were observed at differential relative intensities (reflective of their relative quantities) dependent on the individual. This same methodology can be used to screen for posttranslational modifications and metabolic alterations related to the pathophysiology of T2DM, namely glycated variants of the GcG. FIG. 3 shows spectral overlays of GcG from three individuals (all of genotype Gc-1f/1f), healthy (red), T2DM (green) and id-T2DM (blue). Observed in the spectra originating from the individuals having T2DM are increased levels of signals at 162 Da greater mass than that of the native GcG. This shift in molecular weight corresponds to that expected to result from (non-enzymatic) addition of a 1-deoxyfructosyl adduct, which is consistent with elevated blood glucose levels associated with T2DM. Viewed as groups, the mean level of glycated GcG (integrated ion signals) in the T2DM subjects is ˜4-5-fold greater than that found in the healthy individuals (see FIG. 3 inset).

This example demonstrates both a genetic modification (GM) and a metabolic alteration (MA) present in products stemming from a single gene, and the ability to analyze them simultaneously using a single analysis (i.e., in a single analytical mode).

### Example 5

### Beta-2-microglobulin and Cystatin C

### Metabolic Alterations

In continued population-based screening, glycated variants of two other plasma proteins-beta-2-microglobulin (b2m) (the light chain of the Class I major histocompatibility complex, normally present in plasma at ˜1 mg/L) and cystatin C (cysC) (a cysteine protease inhibitor, normally present in plasma at −0.1 mg/L)—were found at elevated levels in T2DM subjects. Assays were performed by simultaneously extracting b2m and cysC from the same sample preparations used in the GcG assays using extraction pipette tips derivatized with rabbit anti-human b2m and cysC polyclonal IgG. After extraction, non-specifically bound protein was removed through rinsing with HBS, water, 2M ammonium acetate/acetonitrile (3:1 v/v), then water again. Retained protein was next eluted by aspirating 5 μL of matrix solution (2:1 v/v, H2O:ACN saturated with sinapinic acid with 0.4% added TFA) into the tips (covering the solid support) and depositing the matrix/protein mixture onto the surface of a 96-well formatted MALDI-TOF-MS target. Mass spectrometry was performed using a Bruker Autoflex III operating in delayed-extraction linear mode and laser (Nd:YAG) repetition rate of 200 Hz. Spectra (2,500 laser shots) were acquired by summing 25×100 laser-shot spectra [each meeting the criteria of S/N>10 and resolution (FWHM)>1,000] taken from different sites within a sample preparation. Spectra were processed by baseline subtraction followed by signal integration (to baseline) of each signal of interest. For each individual, the relative glycation value (ion signal) was determined by normalizing the integral of the glycated form of the protein (either b2m or cysC) to the integral of all observed forms of the protein.

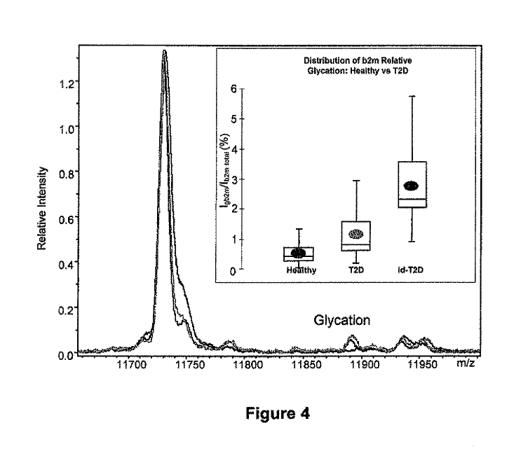

FIG. 4 shows spectral overlays resulting from the b2m MSIA of three individuals, healthy (red), T2DM (green) and id-T2DM (blue). Common to all spectra are signals due to wild-type b2m (m/z=11,730 Da), and matrix adducts (sinapinic acid; at m/z=11,936 & 11,954 Da). Similar to the GcG analyses, increased levels of glycation—indicated by signals at 162 Da greater in molecular weight than b2m—are observed in the spectra originating from the individuals having T2DM. Viewed as groups, the level (relative ion signals) of glycated b2m in the T2DM subjects was 2-5-fold greater than that found in the healthy individuals (FIG. 4 inset).

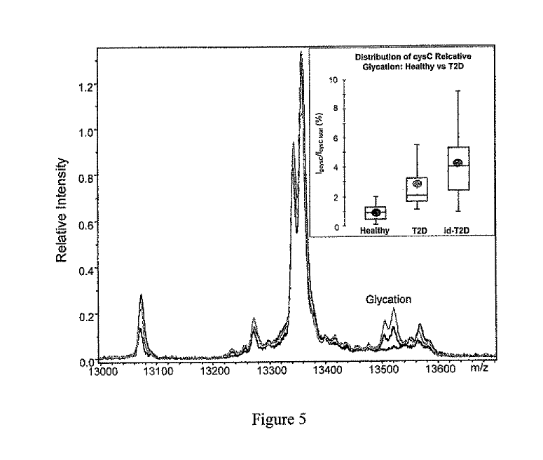

Similar results were obtained during the cysC screening. FIG. 5 shows spectral overlays of cysC and variants produced from the same samples used in the GcG and b2m analyses. Common to all profiles are signals of four forms of cysC: N-terminal desSSP (m/z=13,073), N-term. desS (m/z=13257/13273 w/o or w/hydroxyproline, resp.), native cysC (m/z=13,344) and hydroxyproline cysC (m/z=13360), plus matrix adducts of the wild-type and hydroxyproline cysC (m/z=13550-13584). In addition, signals are observed for glycated cysC (m/z=13509 and 13525) in the diabetic individuals. As with the GcG and b2m, the average values determined for the three subjects show an approximately 3-4 fold relative increase in the glycated signals from the T2DM subjects (FIG. 5 inset).

This example demonstrates the ability to use a single analysis to simultaneously determine multiple forms of products stemming from multiple genes, which include metabolic alterations (MA) related to disease. This example also demonstrates a multiplexed assay able to simultaneously analyze more than one MA related to disease.

### Example 6

### C-Peptide

### Posttranslational Modification

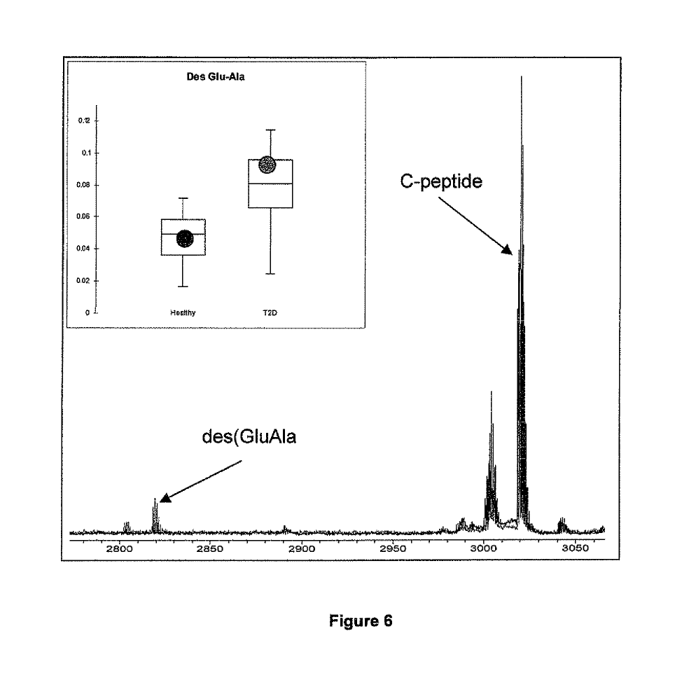

In this study, plasma from Example 1 were qualitatively and semi-quantitatively analyzed for C-peptide using methodologies similar to those used in Examples 2-4. FIG. 6 shows spectra obtained for a healthy individual and an individual suffering from T2DM. Most interestingly, a previously unreported variant of C-peptide, identified as the des(Glu-Ala) isoform, was present in the T2DM population at elevated levels compared to the healthy population, thus establishing a new candidate biomarker—in the form of a PTM—for T2DM (Inset). Without intending to be limited by any particular mechanism, it is believed that dipeptidyl peptidase IV (DPP-IV, CD26, EC 3.4.14.5) is responsible for this particular cleavage product, which is consistent with ongoing research of the pathophysiology of T2DM. This multifunctional transmembrane serine protease can be responsible for the Glu-Ala truncated versions of C-peptide widely seen in this study due to the enzymes specificity to cleave Xaa-Pro or Xaa-Ala from the amino termini of peptide hormones. Taking this into consideration, the substantial increase in relative des(Glu-Ala) C-peptide in the T2DM individuals versus healthy individuals affirms that this specific posttranslationally modified form of C-peptide is an effective biomarker in the clinical diagnosis of T2DM and shows the biological activity of DPP-IV.

This example demonstrates the use of PTM and MA forms of a protein or gene product as direct markers of enzymatic activity related to a disease.

### Example 7

### Enzymatic Signaling

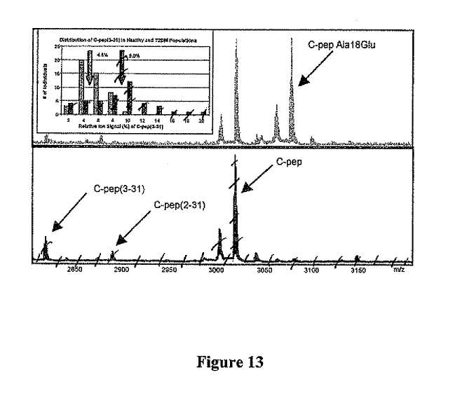

Both glycation and oxidative stress present themselves as positive mass shifts relative to the target proteins. In accordance with the invention, negative mass shifts—i.e., truncations—in certain proteins correlate with T2DM. Briefly, (reflectron) MALDI-TOFMS MSIA assays for C-peptide (C-pep) and insulin (Ins) were developed for use in the studies described here. Upon initial screening in populations, truncated variants of C-pep, insulin and insulin analogs were identified and observed to correlate with the T2DM subject. FIG. 13 shows negative-ion MSIA spectrum qualitatively representative of those obtained for the individuals investigated in this study. Observed in the spectra are intact C-peptide at monoisotopic m/z=3017.50 Da, and signals two other signals registering at m/z=2888.49 Da and 2817.45 Da. With 10 ppm mass accuracy, accompanied by partial sequencing using MALDI-TOF/TOFMS, these signals were identified as C-peptide, C-pep(2-31), and C-pep(3-31), respectively. These three signals were observed universally throughout both the healthy and T2DM subjects. A heterozygous point mutation C-pep Ala18Glu was observed once in the subjects (healthy female). Spectral data from each individual were subjected to relative quantitative analysis by normalizing the ion signal of each qualitatively different species to the total signal from all species. The relative ion signal for each species was then evaluated with respect to the presence of T2DM by grouping data from individuals into their respective subjects. C-pep(2-31) showed little difference between the healthy and T2DM subjects. However, the relative contribution of C-pep(3-31) was found to be comparatively different between the two subjects. FIG. 13 inset shows a histogram comparing the frequency of occurrence between the two subjects for the relative ion signal of C-pep(3-31). A broad distribution averaging ˜9.0% (average of all individuals in the subject) was observed for the T2DM subject, as compared to a narrow distribution averaging ˜4.8% observed for the healthy subject. MSIA spectra of C-peptide from healthy and T2DM (Forward Slash). In FIG. 13, it can be seen that two n-terminal truncated variants, C-pep(2-31) and C-pep(3-31), were observed consistently in both subjects. C-pep(3-31) was observed at higher relative abundance at greater frequency in the T2DM subject (see inset of FIG. 13).

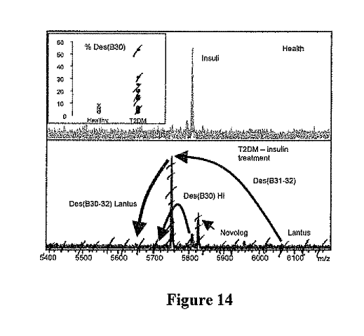

Insulin MSIA was also performed on the subjects. Shown in FIG. 14 are two exemplary spectra taken from a healthy and insulin-dependent T2DM patient (Forward Slash). Intact endogenous insulin is observed to register in both individuals at m/zₐᵥₑ=5,808.4 Da. In addition, insulin homologs of Lantus (insulin glargine; mw=6,063.7) and Novolog (insulin aspart; mw=5831.6) are observed as discrete signals in the T2DM individual (in accordance with his medical records). In accordance with known physiological processing Lantus is observed to degrade initially by the removal of two C-terminal arginine residues, and then a subsequent Thr residue (from the c-terminus of the b-chain). No noticeable degradation products were observed to align with Novolog sequence, however, an endogenous insulin variant was identified (throughout the subjects) as a truncation of the b-chain C-terminal residue (Des(B30) HI). Similar to the C-pep(3-31), this truncated variant was present at higher relative contribution and frequency in the T2DM subject (FIG. 14 inset).

### Example 8

### Transthyretin (a.k.a. Prealbumin or TTR)

### Posttranslational Modification

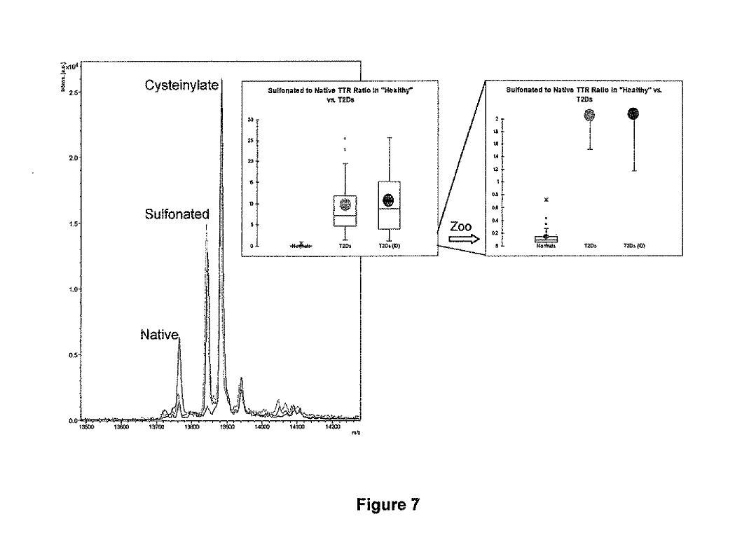

Targeted analysis of intact TTR in the healthy, T2DM and id-T2DM subjects was performed in a manner analogous to that described above for b2m and cysC. FIG. 7 shows mass spectra of TTR from healthy, T2DM and id-T2DM patients in several differentially modified forms, primarily: Native TTR (m/z 13762) Sulfonated TTR (m/z 13842), Cysteinylated TTR (m/z 13881), and Cysteinylglycyl TTR (m/z 13938). As shown in the Inset, findings revealed a dramatic increase in the ratio of sulfonated-to-native TTR in the plasma samples from diabetic patients. Thus, sulfonated TTR serves as an ancillary marker of T2DM by indicating the general degree of inflammation and/or oxidative stress experienced by an individual over the past several days.

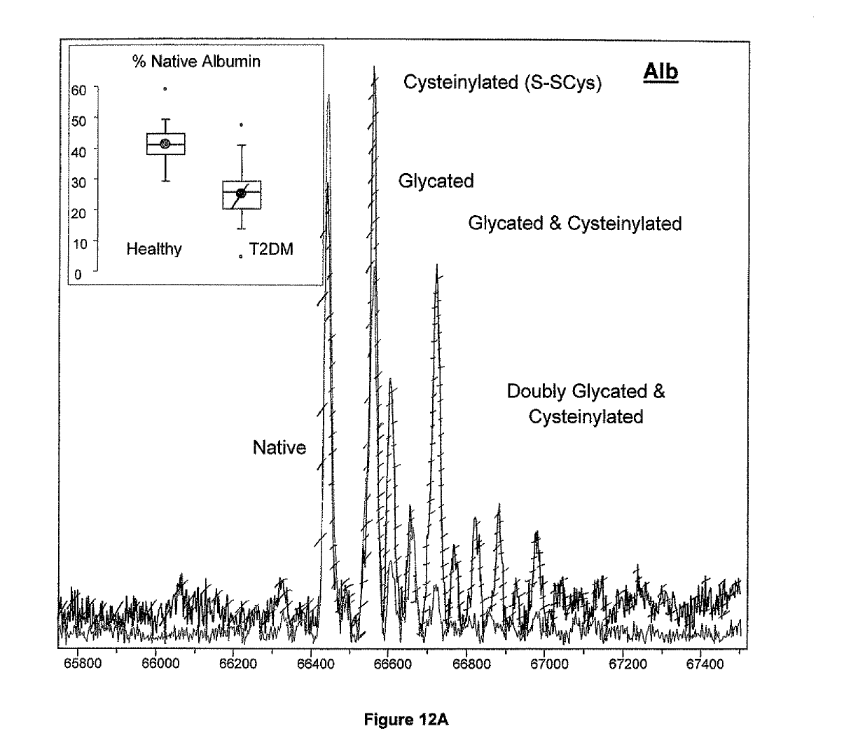

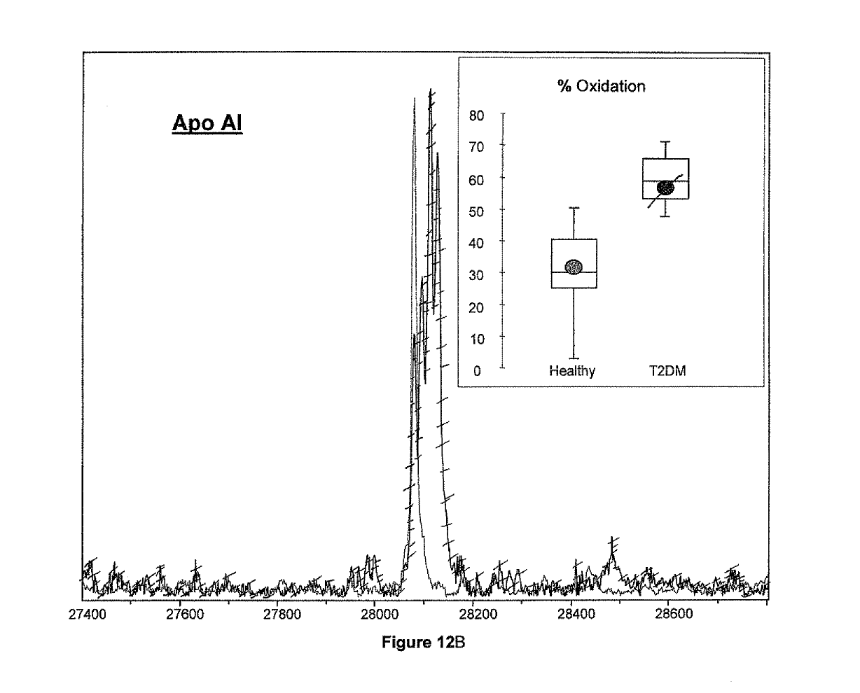

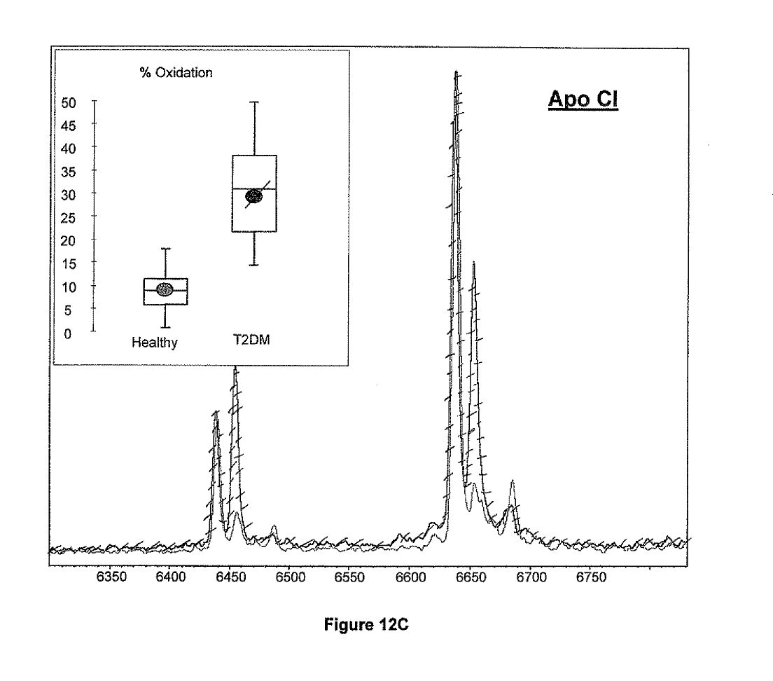

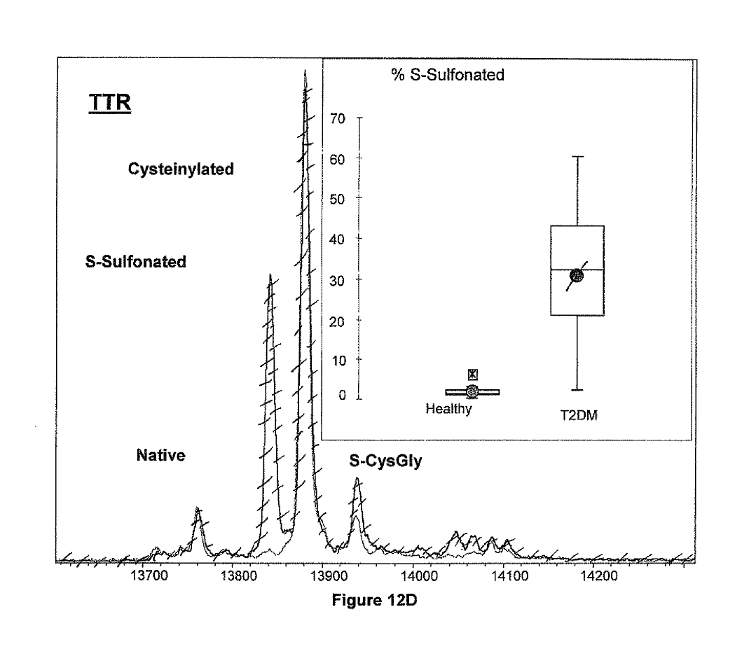

In a manner similar to protein glycation, differential oxidation was observed in a number of proteins. FIGS. 12A-12D illustrate % oxidation (measured per protein as the integral of glycated ion signal normalized to the integral of all species) healthy (n=50) and T2DM (Forward Slash; n=52). FIG. 12A shows overlays of albumin (Alb), FIG. 12B Apolipoprotein A1 (Apo A1), FIG. 12C Apolipoprotein C1 (Apo C1) and FIG. 12D transthyretin (TTR) taken from healthy and T2DM (Forward Slash) individuals. Albumin and TTR exhibit differential oxidation at their free cysteines in the form of, respectively, cysteinylation (dm=119 Da) and sulfonation (dm=80 Da)—(also observed in the albumin spectra are signals due to differential glycation). Oxidation of the apolipoproteins occurred predominantly in the form of sulfoxide formation at the free methionines (three in Apo A1 and one in Apo C1). Also observed in the Apo C1 spectra is a signal due to the truncation of two n-terminal amino acids from the intact species. This example demonstrates the use of PTM forms of a protein as auxiliary biomarker(s) of T2DM.

### Example 9

### Metabolic Alteration Data

### Healthy vs. T2DM Class Modeling

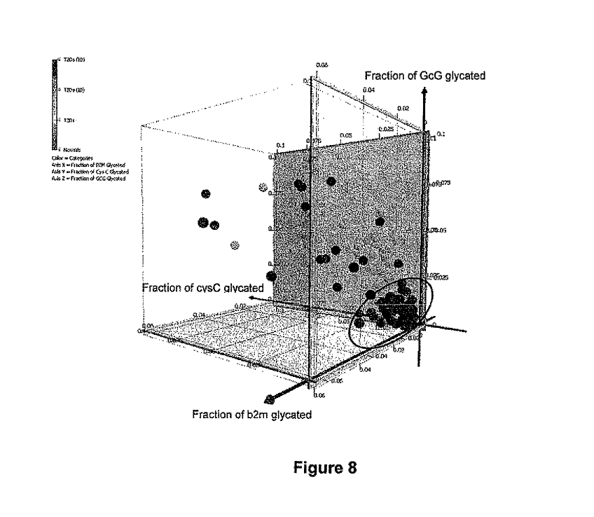

All individuals described in Example 1 were analyzed using the assays described above. A precursory view of MA for GcG, b2m and cysC illustrates the separation of healthy from T2DM individuals in 3-dimensional space (FIG. 8). This separation suggests that with appropriate training, supervised classification techniques may provide an effective means of defining the normal glycation “space” for these three proteins (indicated by oval), which can then be used as a baseline to distinguish abnormal glycation associated with T2DM. To this end, the data from the healthy samples (red dots in FIG. 8) were subjected to principle components analysis (PCA) for the purpose of creating a soft independent modeling of class analogies (SIMCA) classification (using commercially available software: The Unscrambler; Camo Software, Inc., Woodbridge, N.J.).

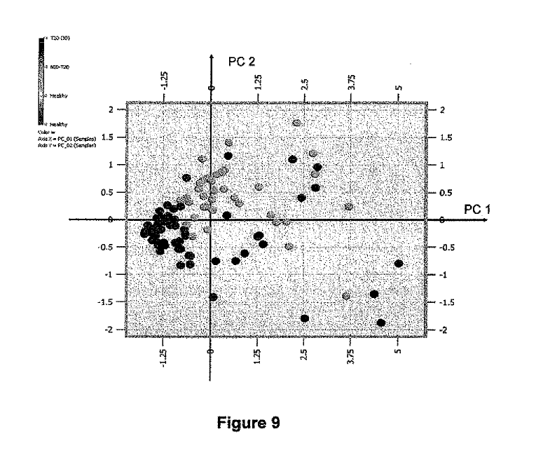

FIG. 9 shows the scores plot of this PCA. Briefly, data from the healthy subject (n=50 individuals; 3-data values per individual) were analyzed with full cross validation and standardized variable variance (i.e., the three glycation values were given equal weight to the model) to establish a model of healthy data. The model was then challenged with data from all subjects (n=102 individuals) to establish its utility in distinguishing healthy from T2DM. Using the model at a significance level of p<0.001, 3 of 50 healthy samples were not classified as healthy, and 2 of 52 T2DM samples were classified as healthy—metrics that equate to a clinical sensitivity and specificity of 96% and 94%, respectively. Noticeably, the degree of separation for the three false positives was observed to be fairly significant, suggesting that these individuals may actually be diabetic without knowing it—i.e., part of the ⅓ of Americans not knowing they have diabetes. Regarding false negatives, it was noted that T2DM is a disease having a “grey area” between healthy and T2DM, typically referred to as pre-diabetic. Once diagnosed as diabetic, achieving this borderline diabetic status is actually a goal for treatment. Thus, good management of T2DM may explain the two false negatives.

In summary, the SIMCA-based analysis of the three glycated proteins shows considerable promise for use in determining and monitoring T2DM, and represents a lead assay suitable for larger-subject challenge. Moreover, it serves as a technical foundation that can be improved with the addition of other markers (once they are found). To fully appreciate this sort of additive approach to biomarker development, it is worth noting that the present invention is not starting by using multivariate analysis to scrutinize large volumes of spectral data that contain both determinate and indeterminate values. Rather, only data from determinate forms of proteins showing promise as markers—in this case, the relative glycation values of plasma proteins—are added to the analysis. In this manner, the value of individual (independent) markers can be evaluated as part of the entire analysis. For instance, the false positive and negative rates reported above (6 and 4%, respectively) were achieved using all three determinants. These metrics are an improvement over using just two of the proteins—e.g., use of only b2m and GcG data resulted in the next-best false positive and negatives rates (of 8 and 12%, respectively). If the contrary was observed, then the non-value marker would have been exclude from the analysis. This approach of “building” a multi-determinant assay is in contrast to examples of clinical proteomics where an abundance of non-targeted spectral data are considered, much of which is not significant to prediction, and in the worst cases cause errors due to spurious appearance in data sets (64, 65). Thus, by eliminating inconsequential (or erroneous) values from the measurement, and adding only determinate data, the present invention contemplates to maximize data and evaluation methods for the accurate classification of disease.

This example demonstrates the use of multiple values of MA, and PCA or class modeling, to accurately detect and diagnose healthy from disease.

### Example 10

### Single Assay

### GcG Genotype and Glycation

An advantage of performing the MS-based GeG assay is that both genotype and protein phenotype (glycation) data can be obtained in a single analysis—each metric independently having value toward T2DM detection and monitoring. Presently there is no single-analysis assay that is capable of producing equivalent data. Using current technologies, for instance, GcG genotyping can be performed at the nucleic acid-level using, e.g., single-nucleotide polymorphism (SNPs) analysis or gene sequencing, Thus, analysis of the three major allelic forms of GcG would require at least two gene-based assays capable of recognizing the two SNP's responsible for the genotypes. Data from such genotyping assays would be combined with glycation data, the assay of which is less straightforward. Similar to HbA1c, measuring the relative abundance of the glycated form of GcG is also important T2DM detection and monitoring. This measurement would require at least two more assays (e.g., protein-based immunometric approaches)—one for all forms of GcG (the denominator) and a second assay capable of recognizing only glycated GcG (the numerator). In total, at least four assays must be performed. Other analytical scenarios may be proposed, but in all cases, multiple assays must be performed to produce the data equivalent of the MS-based assay.

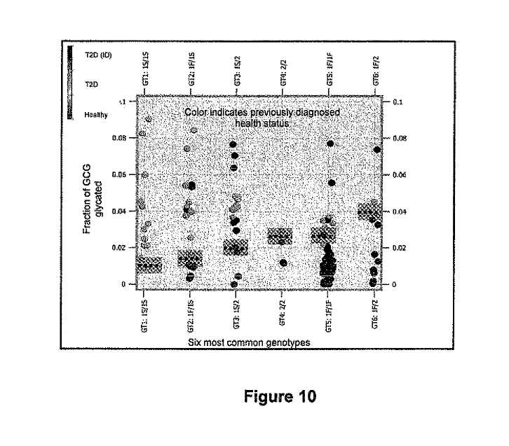

The present invention recognizes using both the GcG genotyping (GM) and glycation (MA) in combination. FIG. 10 shows the results of using both metrics in combination. Each point stems from a single analysis performed on a given individual [healthy (red), T2DM (green) and id-T2DM (blue)]. Defined on the X-axis are the six major genotypes of GcG. Given on the Y-axis is the relative abundance of the glycated GcG found in the individuals. Dashed lines highlighted by gray areas are given to mark reference levels that best separate healthy from T2DM as a function of glycated GcG (versus GcG genotype), and ranges that may indicate individuals adequately managing T2DM (or pre-T2DM). With the exception of a few outliers, there is a genotype-dependent threshold above which glycated GcG levels are indicative of T2DM.

The prospects of this sort of (single-analysis) genotype-protein phenotype assay are significant. Such an assay finds value by: 1) Indicating the likelihood of developing T2DM, 2) Detecting T2DM, and 3) Monitoring the progression (and/or effect of treatment) of T2DM on a personalized level. Referring to the data shown in FIG. 2, the X-axis may be interpreted on its own as the predisposition for T2DM based on genotyping—i.e., the measurement of a genetic risk factor that an individual may develop T2DM within his/her lifetime, with Gc-1s genotypes being more disposed to T2DM. A genotype-dependent threshold for glycation (as an indicator of T2DM) yields a more personalized assay that is able to stratify an individual within the general population based on the initial risk factor as well as the presence of the pathophysiological marker of T2DM—i.e., using the two values in combination to more accurately indicate when an individual has developed T2DM and how he/she is responding to treatment. Such stratification is an essential component of personalized medicine.

This example demonstrates the combined use of GM's and MA's, stemming from a single analysis, to stratify a disease. The single assay and data evaluation method is able to indicate predisposition, onset, progression and response to treatment of diabetes.

### Example 11

### Multimarker

### Time-Dependent Evaluation

A particularly novel use of the data from the different glycated proteins (MA) is to view an individual's blood glucose levels (through the glycation levels of the three proteins) as a function of time, temporal fluctuations in glycation van be viewed by correlation with the in vivo lifetime of the proteins. In addition to the more accurate diagnosis of overt T2DM, other topics of interest here are to more accurately define the “grey shade” of pre-T2DM, as well as to monitor an individual's maintenance of T2DM once it is diagnosed. It is conceivable that individuals can drift in and out of a pre-T2DM (or well-maintained) state within the time points monitored using current markers (immediate and ˜90-days in the past). This effect potentially leads to false readings when an individual is originally screened for diagnosis of T2DM—e.g., a low FGT test (with no OGTT or HbA1c) due extensive fasting prior to testing. The opposite may hold true for individuals already diagnosed with T2DM—e.g., those who periodically skip a treatment or do not adequately fast before a fasting glucose test—potentially leading to an unnecessary change in treatment. Multiplexed assays reflective of different time points in an individual's past may offer some benefits regarding these issues.

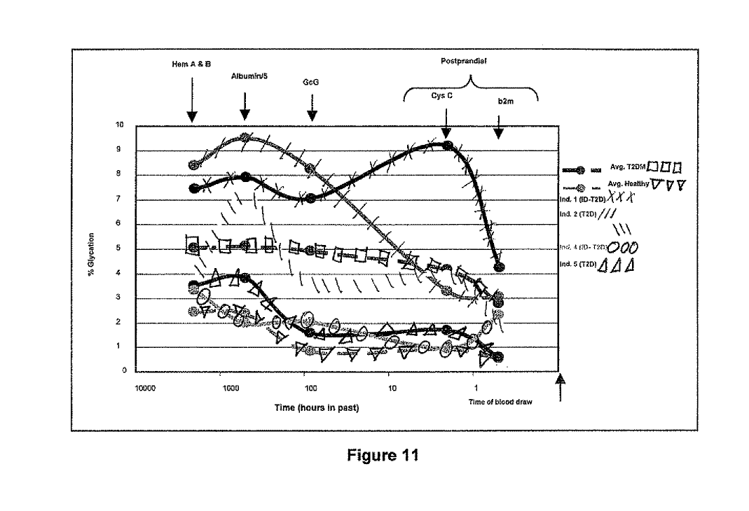

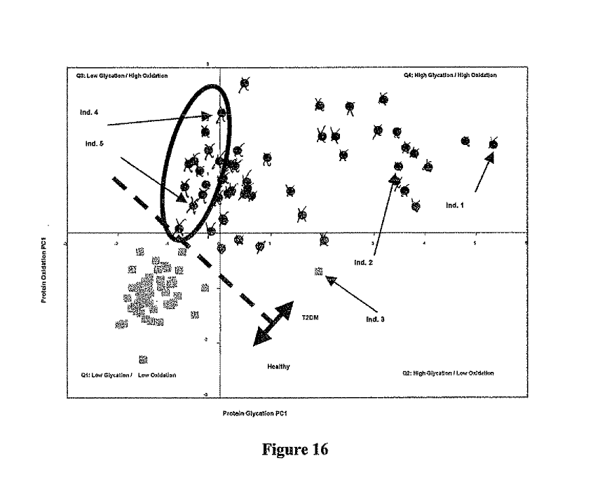

FIG. 11 illustrates the possibility of building a “half-life clock” of the temporal fluctuations in glycation of various proteins. Shown are plots of relative glycation versus time prior to sampling. The in vivo lifetimes of the markers are 0.5, 2, 85, 550 and 2000-hours for b2m, cysC, GcG, Alb and Hem (A&B), respectively. The colored dashed lines link the average values found for the glycated proteins during the analysis of the healthy (Inverted Triangle) and T2DM (Squares) subjects. Also given are data from five individuals indicated in FIG. 16. For Individual 5, all markers are lower than the average values of the respective subject, signifying an adequate and regimented non-insulin based treatment. Individual 4 exhibits roughly the same profile, except with elevated glycation in the most recent past (and with reference to FIG. 16, also exhibits a relatively higher oxidative stress value). At the other extreme, Individual 1 is either not properly administering his treatment, or the treatment itself is not correct. Similarly, 1-2 months into the past, Individual 2 exhibits (extreme) elevated glycation, but within the past week has begun to reduce glycation to a comparatively lower level. Individual 3, not previously diagnosed with T2DM, is observed to fluctuate in and out of the T2DM levels, illustrative of a borderline, or “pre-T2DM” state. Finally, it should be noted that Individual's 3, 4 & 5 all exhibit roughly the same glycation index as measured using glycated hemoglobin, but follow different trajectories in the time leading up to blood draw.

These time-dependent markers allow a detailed view of an individual's glycation status based on the analysis of a single plasma sample. Used as a monitoring tool, the multi-point image provides a detailed picture of an individual's maintenance of T2DM, which is a form of personalized medicine where an individual is monitored longitudinal relative to his/her-self. The multi-point temporal image of healthy glycation serves as the baseline necessary to potentially resolve high-risk individuals (“pre-T2DM”), where it is conceivable that individuals can drift in and out of a T2DM state. Finally, both short- and long-term glycation are monitored simultaneously, which, regarding the “glucose paradox”, is of considerable interest relative to hyperglycemic-induced oxidative stress.

This example demonstrates the use of multiple MA's to view disease management as a function of time.

### Example 12

### Univariate Verification and Multidimensional Analysis

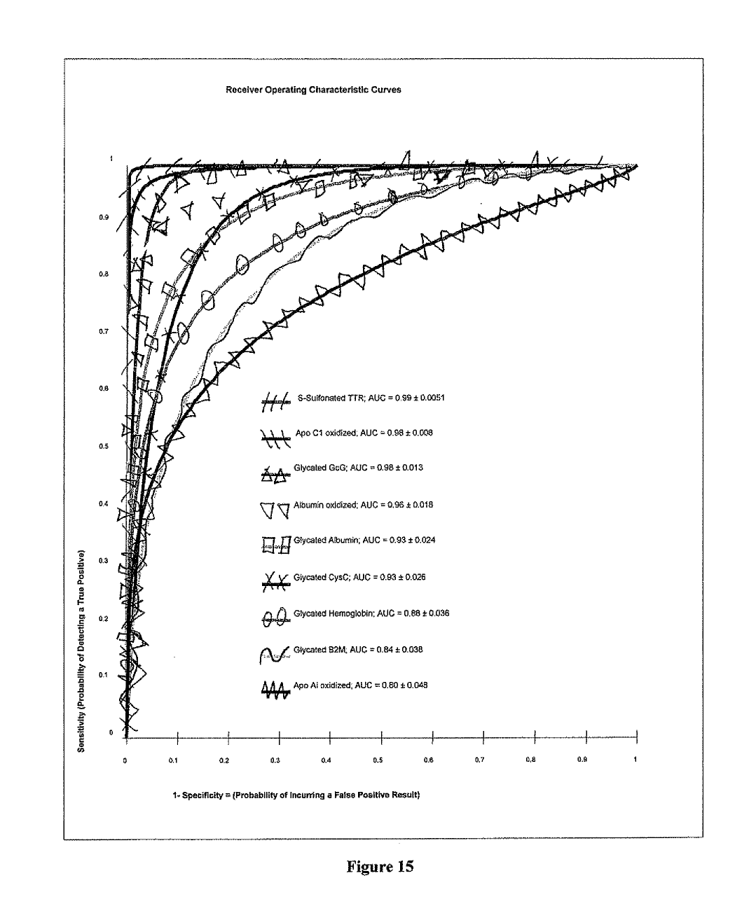

Each glycation and oxidative stress marker was evaluated using receiver operating characteristic (ROC) curves, which reflect the ability of the marker to differentiate healthy from T2DM across all possible assay cutoff values. FIG. 15 shows ROC curves for eight of the markers given in Table 1. Area under the curves ranges from 0.84 to 0.99, demonstrating good separation between the healthy and T2DM subjects. Glycation and oxidative stress are responsible for the proteins variants used in generating the curves.

The data was subjected to principle components analysis (PCA) for the purpose of creating a soft independent modeling of class analogies (SIMCA) classification (using commercially available software: The Unscrambler; Camo Software, Inc., Woodbridge, N.J.). FIG. 16 shows the results of plotting PC1 from glycation data versus PC1 from oxidation data. The healthy individuals cluster in the low-glycation, low-oxidation quadrant—i.e., a quadrant of “healthy” glycation and oxidation, which serves as the point of reference for T2DM diagnosis, as well as is the target for treatment of T2DM once diagnosed. Most of the individuals in the T2DM subjects fall into the high-glycation, high-oxidation quadrant. ˜20% of the T2DM individuals (X's) exhibit relatively good control of glycation, but elevated oxidative stress.

### REFERENCES

1. American Diabetes Association (2008) Economic costs of diabetes in the U.S. in 2007, Diabetes care 31, 1-20.
2. National Diabetes Fact Sheet. http://www.cdc.gov/diabetes/pubs/pdf/ndfs_{—}2005.pdf
3. Feuerstein, B. L, and Weinstock, R. S. (1997) Diet and exercise in type 2 diabetes mellitus, Nutrition (Burbank, Los Angeles County, Calif 13, 95-99.
4. Henry, R. R., and Gumbiner, B. (1991) Benefits and limitations of very-low-calorie diet therapy in obese NIDDM, Diabetes care 14, 802-823.
5. Horton, E. S. (1988) Role and management of exercise in diabetes mellitus, Diabetes care 11, 201-211.
6. Ruderman, N., Apelian, A. Z., and Schneider, S. H. (1990) Exercise in therapy and prevention of type II diabetes. Implications for blacks, Diabetes care 13, 1163-1168.
7. Scheen, A. J. (1998) Aggressive weight reduction treatment in the management of type 2 diabetes, Diabetes & metabolism 24, 116-123.
8. Dawes, J. (2006) The Role of Exercise in the Prevention and Treatment of Gestational Diabetes Mellitus, Strength and Conditioning Journal 28, 66-68.
9. Calvert, M. J., McManus, R. J., and Freemantle, N. (2007) The management of people with type 2 diabetes with hypoglycaemic agents in primary care: retrospective cohort study, Family practice 24, 224-229.
10. Hermann, L. S., Lindberg, G., Lindblad, U., and Melander, A. (2002) Efficacy, effectiveness and safety of sulphonylurea-metformin combination therapy in patients with type 2 diabetes, Diabetes, obesity & metabolism 4, 296-304.
11. Davis, S. N. (2006) Insulin, Oral Hypoglycemic Agents, and the Pharmacology of the Endocrine Pancreas, in Goodman and Gilman's The Pharmacological Basis of Therapeutics (Brunton, L. L., Ed.) 11th ed., McGraw-Hill, New York.
12. Garber, A. J., Ligthelm, R., Christiansen, J. S., and Liebl, A. (2007) Premixed insulin treatment for type 2 diabetes: analogue or human?, Diabetes, obesity & metabolism 9, 630-639.
13. Ahren, B., Simonsson, E., Larsson, H., Landin-Olsson, M., Torgeirsson, H., Jansson, P. A., Sandqvist, M., Bavenhoim, P., Efendic, S., Eriksson, J. W., Dickinson, S., and Holmes, D. (2002) Inhibition of dipeptidyl peptidase IV improves metabolic control over a 4-week study period in type 2 diabetes, Diabetes care 25, 869-875.
14. Ashiya, M., and Smith, R. E. T. (2007) Non-insulin therapies for type 2 diabetes, Nature Reviews: Drug Discovery 6, 777-778.
15. Peters, A. L., and Schriger, D. L. (1998) The new diagnostic criteria for diabetes: the impact on management of diabetes and macrovascular risk factors, The American journal of medicine 105, 15S-19S.
16. Nelson, R. W., Krone, J. R., Bieber, A. L., and Williams, P. (1995) Mass spectrometric immunoassay, Anal Chem 67, 1153-1158.
17. Krone, J. R., Nelson, R. W., and Williams, P. W. (1996) Mass spectrometric immunoassay, Proceedings of SPIE 2680, 415.
18. Intrinsic Bioprobes, Inc. http://www.intrinsicbio.com Tempe, Ariz.
19. Kiernan, U. A., Tubbs, K. A., Gruber, K., Nedelkov, D., Niederkofler, E. E., Williams, P., and Nelson, R. W. (2002) High-throughput protein characterization using mass spectrometric immunoassay, Anal Biochem 301, 49-56.
20. Kiernan, U. A., Addobbati, R., Nedelkov, D., and Nelson, R. W. (2006) Quantitative multiplexed C-reactive protein mass spectrometric immunoassay, J Proteome Res 5, 1682-1687.
21. Kiernan, U. A., Nedelkov, D., and Nelson, R. W. (2006) Multiplexed mass spectrometric immunoassay in biomarker research: a novel approach to the determination of a myocardial infarct, J Proteome Res 5, 2928-2934.
22. Kiernan, U. A., Nedelkov, D., Niederkofler, E. E., Tubbs, K. A., and Nelson, R. W. (2006) High-throughput affinity mass spectrometry, Methods Mol Biol 328, 141-150.
23. Kiernan, U. A., Nedelkov, D., Tubbs, K. A., Niederkofler, E. E., and Nelson, R. W. (2004) Proteomic characterization of novel serum amyloid P component variants from human plasma and urine, Proteomics 4, 1825-1829.
24. Kiernan, U. A., Nedelkov, D., Tubbs, K. A., Niederkofler, E. E., and Nelson, R. W. (2004) Selected expression profiling of full-length proteins and their variants in human plasma, Clin Proteomics 1, 7-16.
25. Kiernan, U. A., Tubbs, K. A., Nedelkov, D., Niederkofler, E. E., McConnell, E., and Nelson, R. W. (2003) Comparative urine protein phenotyping using mass spectrometric immunoassay, J Proteome Res 2, 191-197.
26. Kiernan, U. A., Tubbs, K. A., Nedelkov, D., Niederkofler, E. E., and Nelson, R. W. (2002) Comparative phenotypic analyses of human plasma and urinary retinol binding protein using mass spectrometric immunoassay, Biochem Biophys Res Commun 297, 401-405.
27. Kiernan, U. A., Tubbs, K. A., Nedelkov, D., Niederkofler, E. E., and Nelson, R. W. (2003) Detection of novel truncated forms of human serum amyloid A protein in human plasma, FEBS Lett 537, 166-170.
28. Nedelkov, D. (2006) Mass spectrometry-based immunoassays for the next phase of clinical applications, Expert review of proteomics 3, 631-640.
29. Nedelkov, D., Kiernan, U. A., Niederkofler, E. E., Tubbs, K. A., and Nelson, R. W. (2005) Investigating diversity in human plasma proteins, Proc Natl Acad Sci USA 102, 10852-10857.
30. Nedelkov, D., Kiernan, U. A., Niederkofler, E. E., Tubbs, K. A., and Nelson, R. W. (2006) Population proteomics: the concept, attributes, and potential for cancer biomarker research, Mol Cell Proteomics 5, 1811-1818.
31. Nedelkov, D., and Nelson, R. W. (2006) Surface plasmon resonance mass spectrometry for protein analysis, Methods Mol Biol 328, 131-139.
32. Nedelkov, D., Phillips, D. A., Tubbs, K. A., and Nelson, R. W. (2007) Investigation of human protein variants and their frequency in the general population, Mol Cell Proteomics 6, 1183-1187.
33. Nedelkov, D., Tubbs, K. A., Niederkofler, E. E., Kiernan, U. A., and Nelson, R. W. (2004) High-throughput comprehensive analysis of human plasma proteins: a step toward population proteomics, Anal Chem 76, 1733-1737.
34. Nelson, R. W., Nedelkov, D., Tubbs, K. A., and Kiernan, U. A. (2004) Quantitative mass spectrometric immunoassay of insulin like growth factor 1, J Proteome Res 3, 851-855.
35. Niederkofler, E. E., Tubbs, K. A., Gruber, K., Nedelkov, D., Kiernan, U. A., Williams, P., and Nelson, R. W. (2001) Determination of beta-2 microglobulin levels in plasma using a high-throughput mass spectrometric immunoassay system, Anal Chem 73, 3294-3299.
36. Niederkofler, E. E., Tubbs, K. A., Kiernan, U. A., Nedelkov, D., and Nelson, R. W. (2003) Novel mass spectrometric immunoassays for the rapid structural characterization of plasma apolipoproteins, J Lipid Res 44, 630-639.
37. Tubbs, K. A., Kiernan, U. A., Niederkofler, E. E., Nedelkov, D., Bieber, A. L., and Nelson, R. W. (2005) High-throughput MS-based protein phenotyping: application to haptoglobin, Proteomics 5, 5002-5007.
38. Tubbs, K. A., Kiernan, U. A., Niederkofler, E. E., Nedelkov, D., Bieber, A. L., and Nelson, R. W. (2006) Development of recombinant-based mass spectrometric immunoassay with application to resistin expression profiling, Anal Chem 78, 3271-3276.
39. Tubbs, K. A., Nedelkov, D., and Nelson, R. W. (2001) Detection and quantification of beta-2-microglobulin using mass spectrometric immunoassay, Anal Biochem 289, 26-35.
40. Nelson, R. W. (1999) Sample presentation apparatus for mass spectrometry, (USPTO, Ed.), U.S. Pat. No. 6,004,770.
41. Nelson, R. W. (2000) Mass spectrometer having a derivatized sample presentation apparatus, (USPTO, Ed.), U.S. Pat. No. 6,093,541.
42. Nelson, R. W. (2001) Sample presentation apparatus for mass spectrometry, (USPTO, Ed.), U.S. Pat. No. 6,316,266.
43. Nelson, R. W. (2002) Sample presentation apparatus for mass spectrometry, (USPTO, Ed.), U.S. Pat. No. 6,498,039.
44. Nelson, R. W., and Nedelkov, D. (2003) Bioactive chip mass spectrometry, (USPTO, Ed.), U.S. Pat. No. 6,569,383.
45. Nelson, R. W., and Nedelkov, D. (2005) Bioactive chip mass spectrometry, (USPTO, Ed.), U.S. Pat. No. 6,887,713.
46. Nelson, R. W., Williams, P., and Krone, J. R. (2005) Mass spectrometric immunoassay, (USPTO, Ed.), U.S. Pat. No. 6,974,704.
47. Nelson, R. W., Williams, P., and Krone, J. R. (2007) Mass spectrometric immunoassay, (USPTO, Ed.), U.S. Pat. No. 7,303,888.
48. Tubbs, K. A., Gruber, K. F., and Nelson, R. W. (2004) Integrated high throughput system for the mass spectrometry of biomolecules, (USPTO, Ed.), U.S. Pat. No. 6,783,672.
49. Tubbs, K. A., Gruber, K. F., and Nelson, R. W. (2006) Integrated high throughput system for the analysis of biomolecules, (USPTO, Ed.), U.S. Pat. No. 7,087,165.
50. Tubbs, K. A., Gruber, K. F., and Nelson, R. W. (2007) Integrated high throughput system for the mass spectrometry of biomolecules, (USPTO, Ed.), U.S. Pat. No. 7,311,826.
51. McIntosh, M. (2007) The need to characterize and report the normal heterogeneity of proteins in clinical biological samples, J Proteome Res 6, 2913.
52. Breiman, L. (2001) Random Forests, Machine Learning 45, 5-32.
53. Speeckaert, M., Huang, G., Delanghe, J. R., and Taes, Y. E. (2006) Biological and clinical aspects of the vitamin D binding protein (Gc-globulin) and its polymorphism, Clinica chimica acta; international journal of clinical chemistry 372, 33-42.
54. Cooke, N. E., and Haddad, J. G. (1989) Vitamin D binding protein (Gc-globulin), Endocrine reviews 10, 294-307.
55. Daiger, S. P., Schanfield, M. S., and Cavalli-Sforza, L. L. (1975) Group-specific component (Go) proteins bind vitamin D and 25-hydroxyvitamin D, Proc Natl Acad Sci USA 72, 2076-2080.
56. Ena, J. M., Esteban, C., Perez, M. D., Uriel, J., and Calvo, M. (1989) Fatty acids bound to vitamin D-binding protein (DBP) from human and bovine sera, Biochemistry international 19, 1-7.
57. Williams, M. H., Van Alstyne, E. L., and Galbraith, R. M. (1988) Evidence of a novel association of unsaturated fatty acids with Ge (vitamin D-binding protein), Biochem Biophys Res Commun 153, 1019-1024.
58. Homma, S., Yamamoto, M., and Yamamoto, N. (1993) Vitamin ID-binding protein (group-specific component) is the sole serum protein required for macrophage activation after treatment of peritoneal cells with lysophosphatidylcholine, Immunology and cell biology 71 (Pt 4), 249-257.
59. Yamamoto, N., and Kumashiro, R. (1993) Conversion of vitamin D3 binding protein (group-specific component) to a macrophage activating factor by the stepwise action of beta-galactosidase of B cells and sialidase of T cells, J Immunol 151, 2794-2802.
60. Hirai, M., Suzuki, S., Hinokio, Y., Chiba, M., Kasuga, S., Hirai, A., and Toyota, T. (1998) Group specific component protein genotype is associated with NIDDM in Japan, Diabetologia 41, 742-743.
61. Klupa, T., Malecki, M., Hanna, L., Sieradzka, J., Frey, J., Warram, J. H., Sieradzki, J., and Krolewski, A. S. (1999) Amino acid variants of the vitamin D-binding protein and risk of diabetes in white Americans of European origin, European journal of endocrinology/European Federation of Endocrine Societies 141, 490-493.
62. Malecki, M. T., Klupa, T., Wanic, K., Cyganek, K., Frey, J., and Sieradzki, J. (2002) Vitamin D binding protein gene and genetic susceptibility to type 2 diabetes mellitus in a Polish population, Diabetes research and clinical practice 57, 99-104.
63. Ye, W. Z., Dubois-Laforgue, D., Bellanne-Chantelot, C., Timsit, J., and Velho, G. (2001) Variations in the vitamin D-binding protein (Gc locus) and risk of type 2 diabetes mellitus in French Caucasians, Metabolism: clinical and experimental 50, 366-369.
64. Petricoin, E. F., Ardekani, A. M., Hitt, B. A., Levine, P. J., Fusaro, V. A., Steinberg, S. M., Mills, G. B., Simone, C., Fishman, D. A., Kohn, E. C., and Liotta, L. A. (2002) Use of proteomic patterns in serum to identify ovarian cancer, Lancet 359, 572-577.
65. Baggerly, K. A., Morris, J. S., Edmonson, S. R., and Coombes, K. R. (2005) Signal in noise: evaluating reported reproducibility of serum proteomic tests for ovarian cancer, Journal of the National Cancer Institute 97, 307-309.
66. Characteristics of beta-2-microglobulin. http://web.lfp.cuni.cz/journals/imj/1998/1/415-en.html
67. Filler, G., Bokenkamp, A., Hofmann, W., Le Bricon, T., Martinez-Bru, C., and Grubb, A. (2005) Cystatin C as a marker of GFR-history, indications, and future research, Clin Biochem 38, 1-8.
68. Safadi, F. F., Thornton, P., Magiera, H., Hollis, B. W., Gentile, M., Haddad, J. G., Liebhaber, S. A., and Cooke, N. E. (1999) Osteopathy and resistance to vitamin D toxicity in mice null for vitamin D binding protein, J Clin Invest 103, 239-251.
69. Rifai, N., Gillette, M. A., and Carr, S. A. (2006) Protein biomarker discovery and validation: the long and uncertain path to clinical utility, Nat Biotechnol 24, 971-983.
70. Hermanson, G. T., Krishna Mallia, A., and Smith, P. K. (1992) Immobilized Affinity Ligand Techniques, Academic Press, San Diego, Calif.
71. Anderson, L. (2005) Candidate-based proteomics in the search for biomarkers of cardiovascular disease, J Physiol 563, 23-60.
72. Anderson, N. L., Polanski, M., Pieper, R., Gatlin, T., Tirumalai, R. S., Conrads, T. P., Veenstra, T. D., Adkins, J. N., Pounds, J. G., Fagan, R., and Lobley, A. (2004) The human plasma proteome: a nonredundant list developed by combination of four separate sources, Mol Cell Proteomics 3, 311-326.
73. Jaleel, A., Halvatsiotis, P., Williamson, B., Juhasz, P., Martin, S., and Nair, K. S. (2005) Identification of Amadori-modified plasma proteins in type 2 diabetes and the effect of short-term intensive insulin treatment, Diabetes care 28, 645-652.
74. Nelson, R. W. (1997) The use of bioreactive probes in protein characterization, Mass Spectrometry Reviews 16, 353-376.
75. Bieber, A. L., Tubbs, K. A., and Nelson, R. W. (2004) Metal Ligand Affinity Pipettes and Bioreactive Alkaline Phosphatase Probes Tools for Characterization of Phosphorylated Proteins and Peptides*, Molecular & Cellular Proteomics 3, 266-272.
76. Nelson, R. W., Dogruel, D., Krone, J. R., and Williams, P. (1995) Peptide characterization using bioreactive mass spectrometer probe tips, Rapid Commun Mass Spectrom 9, 1380-1385.
77. Tubbs, K. A., Nelson, R. W., Krone, J. R., and Bieber, A. L. (2005) MASS SPECTRAL STUDIES OF SNAKE VENOMS AND SOME OF THEIR TOXINS, Toxin Reviews 19, 1-22.
78. Nelson, R. W., Lewis, J. K., Dogruel, D., Krone, J. R., and Williams, P. W. (1996) Rapid protein characterization using bioreactive mass spectrometer probe tips, Proceedings of SPIE 2680, 390.
79. Dogruel, D., Williams, P., and Nelson, R. W. (1995) Rapid Tryptic Mapping Using Enzymically Active Mass Spectrometer Probe Tips, Analytical Chemistry 67, 4343-4348.
80. Pribyl, J., and Skladal, P. (2006) Development of a combined setup for simultaneous detection of total and glycated haemoglobin content in blood samples, Biosens Bioelectron 21, 1952-1959.
81. Brancia, F. L., Bereszczak, J. Z., Lapolla, A., Fedele, D., Baccarin, L., Seraglia, R., and Traldi, P. (2006) Comprehensive analysis of glycated human serum albumin tryptic peptides by off-line liquid chromatography followed by MALDI analysis on a time-of-flight/curved field reflectron tandem mass spectrometer, J Mass Spectrom 41, 1179-1185.
82. Elwood, M. (2002) Proteomic patterns in serum and identification of ovarian cancer, Lancet 360, 170; author reply 170-171.
83. Nakhoul, F. M., Miller-Lotan, R., Awaad, H., Asleh, R., and Levy, A. P. (2007) Hypothesis—haptoglobin genotype and diabetic nephropathy, Nature clinical practice 3, 339-344.
84. Quaye, I. K., Ababio, G., and Amoah, A. G. (2006) Haptoglobin 2-2 phenotype is a risk factor for type 2 diabetes in Ghana, Journal of atherosclerosis and thrombosis 13, 90-94.
85. Stephens, J. W., Sozen, M. M., Whittall, R. A., Caslake, M. J., Bedford, D., Acharya, J., Hurel, S. J., and Humphries, S. E. (2005) Three novel mutations in the apolipoprotein E gene in a sample of individuals with type 2 diabetes mellitus, Clin Chem 51, 119-124.
86. Merchant, M., and Weinberger, S. R. (2000) Recent advancements in surface-enhanced laser desorption/ionization-time of flight-mass spectrometry, Electrophoresis 21, 1164-1177.
87. Anderson, N. L., Anderson, N. G., Haines, L. R., Hardie, D. B., Olafson, R. W., and Pearson, T. W. (2004) Mass spectrometric quantitation of peptides and proteins using stable isotope standards and capture by anti-peptide antibodies (SISCAPA), J. Proteome Res 3, 235-244.
88. Sykes, K. F., and Johnston, S. A. (1999) Linear expression elements: a rapid, in vivo, method to screen for gene functions, Nat Biotechnol 17, 355-359.
89. Nelson, R. W., Jarvik, J. W., Taillon, B. E., and Tubbs, K. A. (1999) BIA/MS of epitope-tagged peptides directly from E. coli lysate: multiplex detection and protein identification at low-femtomole to subfemtomole levels, Anal. Chem. 71, 2858-2865.
90. Schmidt, A. M., Hori, O., Brett, J., Yan, S. D., Wautier, J. L., and Stern, D. (1994) Cellular receptors for advanced glycation end products. Implications for induction of oxidant stress and cellular dysfunction in the pathogenesis of vascular lesions, Arteriosclerosis, Thrombosis, and Vascular Biology 14, 1521-1528.
91. Brownlee, M. (1995) Advanced protein glycosylation in diabetes and aging, Annu Rev Med 46, 34.
92. Raj, D. S., Choudhury, D., Welbourne, T. C., and Levi, M. (2000) Advanced glycation end products: a Nephrologist's perspective, Am J Kidney Dis 35, 365-380.

What is claimed:

1. A method for the detection of and monitoring a disease or disorder in a subject, comprising detecting and assaying genetically modified (GM), posttranslationally modified (PTM) or metabolically altered (MA) biomarkers in the subject's body fluid protein.

2. The method of claim 1, wherein the disease or disorder is diabetes.

3. The method of claim 1, wherein the body fluid protein is plasma or urinary protein.

4. The method of claim 1, wherein data obtained from the multiple markers are further evaluated using classification algorithms to establish healthy and diabetic states.

5. The method of claim 1, wherein the biomarkers are correlated with in vivo lifetimes to establish a longitudinal record related to diabetic and pre-diabetic states.

6. The method of claim 1, wherein the biomarkers are correlated with in vivo lifetimes to establish a longitudinal record related to the management and treatment of diabetes.

7. The method of claim 1, wherein the disease or disorder is selected from the group consisting of diabetes, cardiovascular disease, coronary and peripheral artery disease, chronic obstructive pulmonary disease, stroke, cancer, Alzheimer's disease, neuropathy, retinopathy and nutritional deficiencies; either alone or as comorbidities associated with diabetes.

8. The method of claim 1, wherein the biomarker is a glycation biomarker selected from the group consisting of Gc-Globulin(GcG), beta-2-microglobulin (b2m), cystatin C (cysC), Albumin and Hem A&B.

9. The method of claim 1, wherein the biomarker is an oxidation biomarker selected from the group consisting of Albumin, TTR, Apo A1 and Apo C1.

10. The method of claim 1, wherein the biomarker is an enzymatic biomarker selected from the group consisting of C-peptide (C-Pep) and Insulin.

11. A method for the detection of and monitoring a disease or disorder in a subject, comprising determining combinations of GM, PTM and/or MA biomarkers related to the disease or disorder by a multiple assays.

12. A method for the detection and monitoring of a disease or disorder in a subject, comprising determining combinations of GM, PTM and/or MA biomarkers related to the disease or disorder by a single assay.

13. The method of claim 11, wherein the GM, PTM and MA biomarkers are all present on the same gene product and are all detected in a single protein-based analysis.

14. The method of claim 12, wherein the GM, PTM and MA biomarkers are all present on the same gene product and are all detected in a single protein-based analysis.
